(19) United States
(12) Patent Application Publication
(10) Pub. No.: US 2016/0096792 A1
(43) Pub. Date: Apr. 7, 2016

(54) CATALYSTS AND METHODS FOR ALCOHOL DEHYDRATION

(71) Applicant: DOW GLOBAL TECHNOLOGIES LLC, Midland, MI (US)

(72) Inventors: Paul R. Elowe, Midland, MI (US); David G. Barton, Midland, MI (US); Adam Chojecki, Gent (BE); Beata A. Kilos, Midland, MI (US); Dennis W. Jewell, Angleton, TX (US); Adam S. Cieszlak, Bay City, MI (US); Bruce D. Hook, Lake Jackson, TX (US)

(21) Appl. No.: 14/781,144

(22) PCT Filed: Jun. 9, 2014
(86) PCT No.: PCT/US2014/041455

§ 371 Date: Sep. 29, 2015

Related U.S. Application Data
(60) Provisional application No. 61/836,377, filed on Jun. 18, 2013.

(51) Int. Cl. C07C 41/09; B01J 27/06; B01J 27/32; B01J 27/125

(52) U.S. Cl. CPC C07C 41/09; B01J 27/125; B01J 27/06; B01J 27/32

(57) ABSTRACT

Provided is a method for preparing a diaryl ether compound through the dehydration of an aromatic alcohol compound in the presence of a halogenated rare earth element oxide catalyst, wherein the used dehydration catalyst may be regenerated by a halogenation step. The rare earth element oxide is an oxide of a light rare earth element, an oxide of a medium rare earth element, an oxide of a heavy rare earth element, an oxide of yttrium, or a mixtures of two or more thereof.

### FIELD

This invention relates generally to catalysts and methods for the dehydration of aromatic alcohol compounds to ethers. More particularly, the invention uses a halogenated rare earth element oxide catalyst for the dehydration of aromatic alcohol compounds to diaryl ethers.

### BACKGROUND

Diaryl ethers are an important class of industrial materials. Diphenyl oxide (DPO), for instance, has many uses, most notably as the major component of the eutectic mixture of DPO and biphenyl, which is the standard heat transfer fluid for the concentrating solar power (CSP) industry. With the current boom in CSP has come a tightening of the supply of DPO globally and questions surrounding the sustainability of the technology have arisen.

Diaryl ethers are currently manufactured commercially via two major routes: reaction of a haloaryl compound with an aryl alcohol; or gas-phase dehydration of an aryl alcohol. The first route, for example where chlorobenzene reacts with phenol in the presence of caustic and a copper catalyst, typically leads to less pure product and requires high pressure (5000 psig), uses an expensive alloy reactor and produces stoichiometric quantities of sodium chloride.

The second route, which is a more desirable approach, accounts for the largest volume of diaryl ethers produced but requires a very active and selective catalytic material. For instance, DPO can be manufactured by the gas-phase dehydration of phenol over a thorium oxide (thoria) catalyst (e.g., U.S. Pat. No. 5,925,798). A major drawback of thoria however is its radioactive nature, which makes its handling difficult and potentially costly. Furthermore, the supply of thoria globally has been largely unavailable in recent years putting at risk existing DPO manufacturers utilizing this technology. Additionally, other catalysts for the gas-phase dehydration of phenol, such as zeolite catalysts, titanium oxide, zirconium oxide and tungsten oxide, generally suffer from lower activity, significantly higher impurity content and fast catalyst deactivation.

With a chronic shortage of diaryl ethers such as DPO in sight and a pressing need to increase capacity, it has become crucial to develop alternate methods to produce such materials in a cost-effective and sustainable manner.

The problem addressed by this invention, therefore, is the provision of new catalysts and methods for manufacture of diaryl ethers from aryl alcohol compounds.

### STATEMENT OF INVENTION

We have found that halogenated rare earth oxide-based materials are effective catalysts for the preparation of diaryl ethers from aromatic alcohol compounds. Advantageously, the catalysts exhibit remarkable selectivity for the desired product. Moreover, the catalysts can be readily regenerated, thus permitting extended catalyst life. The regeneration step includes feeding a source of halogen atoms, preferably chlorine, to the used catalyst.

In one aspect, therefore, there is provided a method for preparing a diaryl ether compound, the method comprising: providing a reaction vessel having loaded therein a dehydration catalyst comprising a halogenated rare earth element oxide; dehydrating an aromatic alcohol compound over the dehydration catalyst to form a diaryl ether compound; and regenerating the dehydration catalyst by halogenating it with a halogen source.

In another aspect, there is provided a method for regenerating a dehydration catalyst in need of regeneration, the method comprising: providing a dehydration catalyst comprising a halogenated rare earth element oxide, the dehydration catalyst having been used for preparing a diaryl ether compound via dehydration of an aromatic alcohol compound over the dehydration catalyst; and halogenating the dehydration catalyst with a halogen source to regenerate the dehydration catalyst.

### DETAILED DESCRIPTION

Unless otherwise indicated, numeric ranges, for instance as in “from 2 to 10,” are inclusive of the numbers defining the range (e.g., 2 and 10).

Unless otherwise indicated, ratios, percentages, parts, and the like are by weight.

As noted above, the invention provides methods for producing a diaryl ether compound by dehydrating an aromatic alcohol compound in the presence of a dehydration catalyst and regenerating the dehydration catalyst by halogenating with a halogen source.

It has been discovered that dehydration catalysts as described herein exhibit high selectivity for the desired diaryl ether compounds with relatively low formation of undesirable byproducts. For instance, as demonstrated by the examples, in the synthesis of diphenyl oxide from phenol, a selectivity for the DPO of 50% or greater may be achieved. In some embodiments, a selectivity of 80% or greater may be achieved. In some embodiments, a selectivity of 90% or greater, or 95% or greater is possible.

In addition to being highly selective, the catalysts are also advantageous because they are non-radioactive, thus eliminating the safety and environmental issues, as well as higher costs, associated with the handling of radioactive materials, such as the thoria catalysts of the prior art.

The method of the invention comprises: providing a reaction vessel having loaded therein a dehydration catalyst comprising a halogenated rare earth element oxide; dehydrating an aromatic alcohol compound over the dehydration catalyst to form a diaryl ether compound; and regenerating the dehydration catalyst by halogenating it with a halogen source.

The reaction vessel may be any vessel suitable for the reaction steps as described herein and can be, for instance, a batch, semi-batch, plug-flow, continuous-flow, continuous stir type of reactor. The reaction vessel typically is configured so as to enable: control and measurement of temperature, pressure; introduction of ingredients separately or as a mixture; purging thereof by an inert gas (e.g., nitrogen gas); or charging with a reactant gas. When desired, egress of gas therefrom (e.g., excess reaction gas); introduction of the ingredients as a liquid, solid, or slurry; and, in a stirred reactor, rapid stirring of reactor contents via a stir shaft and impeller. A preferred reaction vessel for use in the invention is a vessel loaded with catalyst particles where gaseous reactants are fed into the vessel and flow through the catalyst bed and exit as reaction products.

According to the inventive method, a reaction vessel is provided having loaded therein a dehydration catalyst comprising a halogenated rare earth element oxide. The rare earth element oxide may be an oxide of a light rare earth element, an oxide of a medium rare earth element, an oxide of a heavy rare earth element, an oxide of yttrium, or mixtures of two or more thereof.

By a “light rare earth element” is meant lanthanum, praseodymium, neodymium, or mixtures of two or more thereof. By “oxide of a light rare earth element” is meant a compound that contains at least one oxygen-light rare earth element chemical bond. Examples include lanthanum oxide (La₂O₃), praseodymium oxide (e.g., PrO₂, Pr₂O₃, Pr₆O₁₁, or mixtures), and neodymium oxide (Nd₂O₃).

By a “medium rare earth element” is meant samarium, europium, gadolinium, or mixtures thereof. By “oxide of medium rare earth element” is meant a compound that contains at least one oxygen-medium rare earth element bond. Examples include Sm₂O₃, Eu₂O₃, and Gd₂O₃.

By a “heavy rare earth element” is meant terbium, dysprosium, holmium, erbium, thulium, ytterbium, lutetium, or mixtures thereof. By “oxide of heavy rare earth element” is meant a compound that contains at least one oxygen-heavy rare earth element bond. Examples include, but are not limited to, Tb₂O₃, Tb₄O₇, TbO₂, Tb₆O₁₁, Dy₂O₃, Ho₂O₃, Er₂O₃, Tm₂O₃, Yb₂O₃, and Lu₂O₃.

The rare earth element oxide may also be an oxide of yttrium. By “oxide of yttrium” is meant a compound that contains at least one oxygen-yttrium bond. An example is yttrium oxide (yttria).

In a preferred embodiment of the invention, the rare earth element oxide is yttrium oxide. A particularly preferred halogenated yttrium oxide dehydration catalyst is chlorinated yttrium oxide.

It should be noted that the dehydration catalyst may be loaded in the reactor as the halogenated oxide, or it may be loaded as an oxide or an oxide precursor that is oxidized and/or halogenated within the reactor. Examples of precursors to oxide include, for instance, rare earth element nitrates, acetates, alkanoates, alkoxides, fluorides, chlorides, bromides, iodides, carbonates, hydroxide, or oxalates. Formation of the catalyst within the reactor may, for example, involve heating the precursor at elevated temperature. For instance, heating at 400 to 600° C. is generally sufficient to form the oxide. If the precursor contained a halogen, then the heating at elevated temperature is generally sufficient to provide the halogenated oxide.

Halogenation may also be carried out by contacting the rare earth element oxide with a halogen source such that it undergoes a halogenation reaction. Such contacting may be carried out, for instance, in the gas phase (e.g., chlorine, HCl, or chlorinated organic), liquid phase (e.g., HClaq) or by solid mixing (e.g., NH4Cl) at temperatures ranging, for example, from room temperature to 650° C. For some halogenating sources, such as monochloroethane, elevated temperature is preferred. The dehydration catalyst preferably comprises, in addition to the rare earth element and oxygen, halogen (e.g., chlorine) in an amount of at least 0.001 weight percent, alternatively at least 0.1 weight percent, alternatively at least 1 weight percent, or alternatively at least 2 weight percent. In some embodiments, the dehydration catalyst may comprise halogen (e.g., chlorine) in an amount of less than 50 weight percent, alternatively 40 weight percent or less, alternatively 30 weight percent or less, alternatively 20 weight percent or less, alternatively 10 weight percent or less, or alternatively 2 weight percent or less.

The preparation of the dehydration catalyst may be carried out such that it provides a BET surface area that is sufficiently high as to enable a commercially viable product yields. Synthesis methods known to those skilled in the art may be performed to maximize the active surface area that selectively produces the desired product. These methods include, but are not limited, to sol-gel preparations, flame pyrolysis, colloidal routes, templating approach and milling. Additionally, compounds may be added to increase surface area such as, but not limited to, sacrificial porogens, structure-directing compounds, exfoliating agents, and/or pillaring agents. The dehydration catalyst preferably has a BET surface area greater than 5 m²/g, more preferably greater than 50 m²/g, and further preferably greater than 150 m²/g.

The dehydration catalyst in the reaction vessel may optionally contain a binder and/or matrix material that is different from the oxide of the rare earth element. Non-limiting examples of binders that are useful alone or in combination include various types of hydrated alumina, silicas and/or other inorganic oxide sols, and carbon. Upon heating, the inorganic oxide sol, preferably having a low viscosity, is converted into an inorganic oxide binder component.

Where the dehydration catalyst contains a matrix material, this is preferably different from the rare earth element oxide and any binder. Non-limiting examples of matrix materials include clays or clay-type compositions.

The dehydration catalyst, including any binder or matrix materials, may be unsupported or supported. Non-limiting examples of suitable support materials include titania, alumina, zirconia, silica, carbons, zeolites, magnesium oxide, and mixtures thereof. Where the dehydration catalyst contains a binder, matrix or support material, the amount of halogenated rare earth element oxide (the active component of the catalyst) may be between 1 and 99 percent by weight based on the total weight of the catalyst (including the halogenated oxide, and any support, binder or matrix materials).

The dehydration catalyst may be subjected to a calcination step prior to use by heating at elevated temperature. Such calcination may render the catalyst more active and/or selective. In some embodiments, calcination is carried out by heating the material at a temperature of 200° C. or greater, alternatively 400° C. or greater, alternatively 450° C. or greater, or alternatively 500° C. or greater. While there is no specific upper limit on the calcination temperature, the material should be calcined at a temperature below the temperature at which the halide begins to decomposes back to the oxide. Such heating may be continued, for instance, for 30 minutes to 1 hour or more.

The dehydration catalyst may be formed into various shapes and sizes for ease of handling. For instance, the catalyst (plus any binder, matrix, or support) may be in the form of pellets, spheres, or other shapes commonly used in the industry.

According to the process of the invention, an aromatic alcohol compound is dehydrated over the catalyst in order to form a diaryl ether compound. Suitable aromatic alcohol compounds include aromatic compounds containing at least one alcohol group and one, two, three or more aromatic moieties. Examples of compounds include phenols and α- and β-hydroxy-substituted fused aromatic ring systems. Apart from the hydroxy substituent, the compounds may be unsubstituted, as in phenol or naphthol. Optionally, however, the compounds may be further substituted with at least one alkyl group containing from 1 to about 10 carbon atoms, preferably, from 1 to 3 carbon atoms, or substituted with at least one alternative substituent which is inert to the dehydration coupling reaction. Suitable inert substituents include cyano, amino, nitro, carboxylic acid (e.g., COOH or C₁-C₆—COOH), ester, C₆-C₁₂ aryl, C₂-C₆ alkenyl, alkyloxy, aryloxy, and phenoxy moieties. It is also possible for the aromatic alcohol compound to be substituted with both an alkyl substituent and one of the alternative inert substituents. Each of the aforementioned alkyl substituents and/or alternative inert substituents is attached preferably to an aromatic ring carbon atom which is located in an ortho, meta or para position relative to the hydroxy moiety. Optionally, the alkyl substituent may contain from 3 to 4 carbon atoms, and in combination with a phenol or fused aromatic ring system may form a saturated ring fused to the aromatic ring. An acceptable feed may contain a mixture of aromatic alcohols, including mixtures of the foregoing.

Non-limiting examples of suitable phenols include unsubstituted phenol, m-cresol, p-cresol, 3,4-xylenol, 3,5-xylenol, and 3,4,5-trimethylphenol. Other suitable phenols include compounds corresponding to the above-mentioned examples except that one or more of the methyl substituents are replaced by an ethyl, propyl or butyl substituent. Non-limiting examples of α- and β-hydroxy-substituted fused aromatic ring systems include α- and β-naphthol and 5-tetralinol. Other non-limiting examples of aromatic alcohols include benzenediols (catechol, resorcinol or hydroquinone), o-cresol, o-phenylphenol, m-phenylphenol or p-phenylphenol. One skilled in the art may find other phenols and α- and β-hydroxy-substituted fused aromatic ring systems which are also suitable for the purposes of this invention. Preferably, the aromatic alcohol is unsubstituted phenol or a substituted phenol wherein the substituent is methyl, ethyl or hydroxyl. More preferably, the aromatic alcohol is unsubstituted phenol, cresol or a benzenediol. Most preferably, the aromatic alcohol is unsubstituted phenol.

According to the method of the invention for preparing a diaryl ether, a catalyst as described herein is contacted with the aromatic alcohol compound. The contacting of the catalyst with the aromatic alcohol compound is carried out under reaction conditions such that the diaryl ether is formed.

The catalyst is contacted with the aromatic alcohol compound either in the gas phase or in the liquid phase. In addition, the aromatic alcohol may be diluted with a diluent or it may be neat. Suitable diluents include, without limitation, nitrogen, argon, water vapor, water, oxygen or hydrogen. When a diluent is used, the concentration of the aromatic alcohol compound may be, for instance, 1 volume percent or greater and less than 100 volume percent.

In a preferred embodiment, the aromatic alcohol is contacted with the catalyst in the gas phase. Typically, the aromatic alcohol is introduced into a reactor containing the catalyst at elevated temperature, for instance, between 200 and 800° C., alternatively between 300 and 600° C., alternatively between 400 and 600° C., or alternatively between 450 and 550° C. The reaction may be conducted at atmospheric pressure, under reduced pressure, or at elevated pressure such as up to 5000 psi. In some embodiments, atmospheric pressure or slightly above (e.g., up to about 50 psi) is preferred. In some embodiments, the gas flow rate of the aromatic alcohol over the catalyst (weight hourly space velocity or WHSV) is from 0.01 to 100 grams per gram of catalyst per hour (g/g·h). In some embodiments, WHSV is from 0.1 to 20 g/g·h, alternatively 0.1 to 5 g/g·h, or alternatively 0.1 to 1 g/g·h.

In some embodiments, it may be useful to subject the reactor to startup conditions which may provide various benefits, such as prolonging catalyst life. Suitable startup conditions include, for example, exposing the catalyst to dilute amounts of the aromatic alcohol at lower temperature before changing to full operating conditions as described above and demonstrated by the examples.

As the alcohol dehydration reaction progresses, the dehydration catalyst tends to lose some of its activity. In the invention, therefore, the dehydration catalyst is regenerated, which serves to boost the activity of the catalyst allowing it to continue efficiently dehydrating an aromatic alcohol compound to a diaryl ether compound. Regeneration in the invention process is carried out by halogenating the catalyst with a halogen source.

Halogen sources suitable for use in the invention include any materials capable of providing a reactive halogen atom, e.g., chlorine or fluorine, with chlorine atoms being preferred. The halogen source may be a solid, liquid or gas, but preferably it is a gas when contacted with the oxide. The gaseous state may be achieved, for instance, by using a halogen source that is already gaseous at room temperature and pressure, or by vaporizing an otherwise non-gaseous material at the appropriate temperature and/or pressure. Examples of halogen sources include, without limitation, chlorinated organic and/or inorganic compounds or fluorinated organic and/or inorganic compounds. More specific examples include, without limitation, monochloroethane, ammonium chloride, hydrogen chloride, ammonium fluoride, carbon tetrachloride, methyl chloride, methylene chloride, chloroform, chlorine gas, dichloroethane, trichloroethane, tetrachloroethane, other higher halogenated organics, etc.

Typically, the halogenation is conducted by contacting the used catalyst with the halogen source. Such contacting may be carried out, for instance, at temperatures ranging from room temperature to 650° C. For some halogenating sources, such as monochloroethane, elevated temperature is preferred. The halogen source may be fed into the reactor periodically to regenerate the catalyst, or it may be fed continuously for continuous regeneration. Moreover, the halogen source may be fed separately from or concurrently with the other steps of the process. For instance, the halogen source may be fed along with the aromatic alcohol. This latter embodiment, may be particularly suitable where the process is run in a continuous mode. When halogenation is conducted as a separate step, it may be desirable in some embodiments to purge the reactor with inert gas, such as nitrogen, prior to feeding the halogenation source to the used catalyst. To reduce downtime, halogenation may, for instance, be conducted in a two or more reactor swing operation mode. Thus, for example, one reactor containing depleted catalyst may be subjected to halogenation and a second reactor, containing regenerated catalyst, used for the dehydration reaction. When the catalyst in the second reactor is depleted and the catalyst in the first has undergone the halogenation, the reactors can be switched.

In some embodiments, halogenation is conducted until a regenerated catalyst is achieved that comprises, in addition to the rare earth element and oxygen, halogen (e.g., chlorine) in an amount of at least 0.001 weight percent, alternatively at least 0.1 weight percent, alternatively at least 1 weight percent, or alternatively at least 2 weight percent. In some embodiments, the regenerated catalyst may comprise halogen (e.g., chlorine) in an amount of less than 50 weight percent, alternatively 40 weight percent or less, alternatively 30 weight percent or less, alternatively 20 weight percent or less, alternatively 10 weight percent or less, or alternatively 2 weight percent or less.

It should be noted that there is no particular requirement in the invention that a catalyst achieve a certain loss of activity before it can be regenerated. Indeed, as described below, regeneration can simply be carried out by feeding the halogenation gas into the reactor along with the aromatic alcohol. In some embodiments, however, it may be desirable to begin the regeneration process once the dehydration has lost, for example, 20 percent or more, alternatively 40 percent or more, of its activity (as measured by a reduced rate of conversion of aromatic alcohol). Other actions may trigger a desire to regenerate the catalyst including, for instance, if the maximum temperature of the reactor is reached or selectivity is reduced.

In addition to halogenation, the catalyst may optionally further be regenerated by decoking. Decoking is typically conducted by oxidizing the catalyst in the presence of an oxygen containing gas, such as air, at elevated temperature. For instance, heating at 200° C. or greater, preferably 400° C. or greater, and up to 650° C., is generally sufficient for the decoking/oxidation. In some cases, higher temperatures may be used, e.g., up to 1000° C. The amount of time is not critical and may, for instance, range from 1 hour or shorter to 100 hours or longer. By way of specific example, if the catalyst is based on yttria, oxidation at 200° C. to 600° C. is typically suitable. The oxidation of carbonaceous deposits (decoking) step may be carried out before, after, and/or concurrently with, the halogenation step.

The method of the invention may be carried out as a batch-wise or as a continuous process and the order of the various steps may be interchanged, as would be understood by a person of ordinary skill in the art. For example, as noted above, regeneration may be carried out as a separate step following dehydration reaction, or it may be conducted concurrently with the dehydration reaction. In addition, the diaryl ether product may be removed from the reaction periodically, or it may be recovered continuously.

The diaryl ether product formed in the process of the invention is recovered from the catalyst and optionally further purified. Unreacted alcohol and other reaction by-products may be separated using methods known in the art and, in the case of the unreacted alcohol, may optionally be recycled to the reaction. Recovery and purification methods include but are not limited to condensation, distillation, crystallization (e.g., crystal refining), and simulated moving bed technique or a combination thereof.

By way of specific, non-limiting, example that may be particularly suitable for a liquid reaction product or condensed reaction product (e.g., diphenyl ether), the following procedure may be followed. The crude product may be collected in a settling/storage drum or tank as feed forward to distillation. The storage drum may be designed to capture catalyst fines that escape the reactor with the crude product. Additional techniques for removing fines, such as filtering, may also be used. Liquid from the storage drum may be fed through filters to the crude distillation tower where unreacted aromatic alcohol (e.g., phenol) and water are stripped to the tower overheads and raw diaryl (e.g., diphenyl) ether and heavies are removed from the tower bottoms. Gas phase feed from the reactor to the separation system is also possible with appropriate management of catalyst fines from the reactor. A distillation tower with the capability for both stripping and rectification is preferred. However, the system can be operated in stripping service only. The tower can recover unreacted aromatic alcohol and water from the overheads of the tower and forward raw diaryl ether and other heavy impurities to the product finishing tower. This crude distillation tower may operate at approximately the following conditions when used for phenol/diphenyl ether: 40 mmHg absolute pressure, 185° C. bottoms temperature and 100° C. condensing temperature. The liquid from the overheads of the crude distillation tower may be sent to an aromatic alcohol drying tower. The function of the drying tower is to separate the water from unreacted aromatic alcohol and recycle the aromatic alcohol back to the reaction vessel for use in accordance with the inventive process. The distillate of the drying tower, primarily water containing between 0.1 and 20 wt. % aromatic alcohol, may be sent to treatment or to additional recovery steps—such as solvent extraction/distillation. The drying tower may be operated at approximately the following conditions (e.g., where the aromatic alcohol is phenol): 1 psig pressure, 183.5° C. bottoms temperature and 115° C. condensing temperature.

The liquid from the bottoms of the crude distillation tower may be sent to a product finishing tower. The function of the product finishing tower is to separate the diaryl ether product from heavy impurities. The distillate of the product finishing tower, the diaryl ether, may be sent to storage. The tower bottoms is primarily heavies which may be disposed of. The product finishing tower operates at approximately the following conditions (e.g., when the diaryl ether is diphenyl ether): 30 mmHg absolute pressure, 188.6° C. bottoms temperature and 155° C. condensing temperature.

The distillation scheme described in the example above is for illustration only and is not intended to limit the invention. Other distillation sequences and/or separation technologies, such as crystallization, simulated moving bed techniques, use of a flash vessel, etc., may be employed to more effectively utilize assets for the most cost economical diaryl ether production. If distillation is used for all separation steps, five distillation sequences may be advantageously used to separate the main components in the crude diaryl ether: typically water, aromatic alcohol, diaryl ether and heavies. The actual sequence can be selected to best match the equipment and utility conditions available. The indicative sequence presented above recovers aromatic alcohol within the first two steps in order to facilitate its recycle to the reactor without intermediate storage—due to the relatively high volume of aromatic alcohol. However, given the appropriate equipment, any of the five sequences may be used to recover and recycle the aromatic alcohol and purify the diaryl ether product while rejecting the two by-product streams. In some cases, crystallization purification may be an advantaged alternative if appropriate facilities are available. Impurities can be efficiently excluded during diaryl ether crystallization, and high purity product can be produced at lower energy consumption and moderate conditions compared to distillation requirements. Although it can be used for more gross separation of the diaryl ether product, crystallization is most cost effective, compared to distillation, to complete the final stages of purification where the highest purities are encountered. A number of different sequences are possible for integrating crystallization in the diaryl ether separation scheme. It may be practical to utilize crystallization or a combination of crystallization-distillation after the bulk aromatic alcohol and water fraction have been distilled from the crude mixture. Single or multi-stage crystallization or a hybrid crystallization-distillation sequence can then be used to efficiently produce the diaryl ether product. Optimization of recycle streams between staged crystallizers or, similarly, between a crystallizer and distillation system may result in an efficient diaryl ether purification.

In some cases, such as when the aromatic alcohol is phenol, a water-phenol azeotrope may result in a process water stream that contains significant amounts of phenol. Liquid-liquid extraction coupled with solvent recovery by distillation is one technique that may be used for recovering the aromatic alcohol from water that may be used to improve aromatic alcohol recovery and reject a water stream of significantly lower aromatic alcohol content. Recovery of this aromatic alcohol can lower feedstock costs. While toluene is an effective solvent for phenol-water separation, other effective solvents are possible.

Through use of appropriate purification techniques, including those described above, very pure diaryl ether products may be achieved, for instance, greater than 99% purity, or greater than 99.9% purity, or even greater than 99.99% purity. Note that the purification system can be made suitable for removing halogenated impurities from the diaryl ether product if needed and so desired.

The methods for introducing the reactants and the regeneration/decoking agents, either continuous or periodic, are well known to one of ordinary skill in the art. For instance, the regeneration agents may be introduced using the same apparatus as the reactant feed system, or may be a separate, dedicated feed system as is most appropriate for the particular agents and regeneration conditions.

In the case of periodic catalyst regeneration and/or decoking, the effluent from said treatment may be diverted to process equipment other than the purification train to suitably treat the effluent. A person of ordinary skill in the art will recognize that treatment options for this effluent stream include but are not limited to condensers, scrubbers, adsorbers, thermal treatment units, oxidation units and similar apparatus or combination of apparatus.

In some embodiments, the diaryl ether prepared by the process of the invention is diphenyl oxide (DPO). Other diaryl ether compounds that may be prepared by the process of the invention include, without limitation, compounds containing at least one ether functionality whereby two aryl moieties are connected by an oxygen atom (Ar—O—Ar′), including polyaryl compounds and compounds prepared from the aromatic alcohols described above. Specific examples include, but are not limited to, phenoxytoluene isomers, including 3-phenoxytoluene, ditolyl ether isomers, polyphenyl ethers (PPEs), biphenylyl phenyl ether isomers and naphthyl phenyl ethers.

The diaryl ethers prepared by the invention are useful in a variety of applications, including as high temperature solvents, as intermediates in preparing flame retardants and surfactants, and as components in heat transfer fluids. Furthermore, certain diaryl ethers prepared by the invention are useful as high performance lubricants and as intermediates in preparing pyrethroid insecticides.

In some embodiments, a preferred use of the diaryl ether is in high temperature heat transfer fluids. High temperature heat transfer fluids may be prepared by making the diaryl ether according to the process described above and then mixing the diaryl ether with biphenyl. The amounts necessary to provide a suitable fluid can be readily determined by a person with ordinary skill in the art. For diphenyl oxide and biphenyl, the amount of DPO may be, for instance, from 70 to 75 weight percent based on the total weight of the DPO and biphenyl. A preferred amount of DPO is that required to form a eutectic mixture with the biphenyl, which is about 73.5 weight percent based on the total weight of the DPO and biphenyl.

Some embodiments of the invention will now be described in detail in the following Examples.

### EXAMPLES

### Example 1

The synthesis of lanthanum oxychloride is carried out by thermal decomposition of LaCl₃.7H₂O. A sample of the powdered precursor (approximately 10 g) is calcined in air in a static calcination oven under the following temperature protocol: ramp 1.41° C./min to 550° C., dwell 3 hrs at 550° C., cool down to room temperature. The elemental composition of the catalyst is assayed by X-ray fluorescence spectroscopy (XRF) to 17.23 wt. % chlorine, 69.63 wt. % lanthanum and 13.14 wt. % oxygen (balance). Thus, the elemental composition of the catalyst is La_{1.00}O_{1.64}Cl_{0.97}. The specific surface area (BET) of the catalyst sample is measured to 6.2 m²/g and its pore volume to 0.013 cm³/g. The XRD data shows the presence of lanthanum oxychloride phases.

### Example 2

The lanthanum oxychloride catalyst from Example 1 is used for the dehydration of phenol. The powder is pressed and sieved to obtain particles that are between 0.60 mm and 0.85 mm in diameter. The particles are loaded into an electrically heated stainless steel reactor tube and heated to the reaction temperature with nitrogen flowing through the tube. After the reaction temperature is reached, vapor-phase phenol is passed through the reactor tube. The conversion of phenol is carried out at a weight hourly space velocity of 1 (WHSV=gram phenol/gram catalyst-hour) and at 500° C. Test conditions and results are shown in Table 1.

TABLE 1

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 0.70% | 95.86% | 0.02% | 4.12% | 0.00% | 0.00% | 0.00% |
| Feed: PhOH | | | | | | | |
| ToS = 1.5 hrs | | | | | | | |
| WHSV 1 hr⁻¹ | | | | | | | |
| T = 500° C. | 0.82% | 95.83% | 0.07% | 4.09% | 0.00% | 0.00% | 0.00% |
| Feed: PhOH | | | | | | | |
| ToS = 2.75 hrs | | | | | | | |
| WHSV 1 hr⁻¹ | | | | | | | |
| T = 500° C. | 0.82% | 95.90% | 0.18% | 3.93% | 0.00% | 0.00% | 0.00% |
| Feed: PhOH | | | | | | | |
| ToS = 3.75 hrs | | | | | | | |
| WHSV 1 hr⁻¹ | | | | | | | |
| T = 500° C. | 0.74% | 95.70% | 0.13% | 4.17% | 0.00% | 0.00% | 0.00% |
| Feed: PhOH | | | | | | | |
| ToS = 7.5 hrs | | | | | | | |
| WHSV 1 hr⁻¹ | | | | | | | |

OPP: orthophenylphenol.
DBF: dibenzofuran.
O-BIPPE: ortho-biphenylphenyl ether.
M-BIPPE: meta-biphenylphenyl ether.
P-BIPPE: para-biphenylphenyl ether.
PhOH: phenol.
N2: nitrogen.
ToS: time on stream (ToS = 0 hours defined at start of phenol flow).

### Example 3

A 1M PrCl₃ solution, prepared by dissolving 10 g PrCl₃ in 50 mL DI H₂O, is added dropwise along with tetrapropylammonium hydroxide (76.36 g) over 15 min into a 600 mL beaker containing an initial 100 mL DI H₂O. The solution is stirred at 500 rpm on magnetic stir plate with a 4.5 inch stir bar. The resulting green precipitate is allowed to age in solution for 1 h with stirring, after which it is centrifuged at 5000 rpm for 10 min. The decanted precipitate is placed into an oven, dried at 120° C. for 4 h and calcined at 500° C. for 4 h with a ramp rate of 5° C./min to yield approximately 8 g of product. Neutron activation analysis reveals a total chlorine concentration of 1.17 wt %.

The catalyst is evaluated using a similar procedure as in Example 2. Test conditions and results are shown in Table 2.

TABLE 2

| | Conversion | Selectivity [mol. %] | | | | | |
|---|---|---|---|---|---|---|---|
| | [mol. %] | Diphenyl | | | | | |
| Test Conditions | Phenol | Oxide | OPP | DBF | O-BIPPE | M-BIPPE | P-BIPPE |
| T = 500° C. | 2.63% | 73.15% | 1.94% | 24.91% | 0.00% | 0.00% | 0.00% |
| Feed: PhOH | | | | | | | |
| ToS = 1 hr | | | | | | | |
| WHSV 1 hr⁻¹ | | | | | | | |
| T = 500° C. | 2.75% | 78.64% | 2.16% | 19.20% | 0.00% | 0.00% | 0.00% |
| Feed: PhOH | | | | | | | |
| ToS = 2 hrs | | | | | | | |
| WHSV 1 hr⁻¹ | | | | | | | |
| T = 500° C. | 3.70% | 76.14% | 1.92% | 21.94% | 0.00% | 0.00% | 0.00% |
| Feed: PhOH | | | | | | | |
| ToS = 3.5 hrs | | | | | | | |
| WHSV 1 hr⁻¹ | | | | | | | |
| T = 500° C. | 2.28% | 73.28% | 3.58% | 23.14% | 0.00% | 0.00% | 0.00% |
| Feed: PhOH | | | | | | | |
| ToS = 5.75 hrs | | | | | | | |
| WHSV 1 hr⁻¹ | | | | | | | |

### Example 4

A 1M NdCl₃ solution, prepared by dissolving 17.94 g NdCl₃ in 50 mL DI H₂O, is added dropwise along with tetrapropylammonium hydroxide (76.26 g, from a 40 wt % TPAOH solution) over 15 min into a 600 mL beaker containing an initial 100 mL DI H₂O. The solution is stirred at 500 rpm on magnetic stir plate with a 3 inch stir bar. The resulting precipitate is allowed to age in solution for 1 h with stirring, after which it is centrifuged at 5000 rpm for 10 min. The decanted precipitate is placed into an oven, dried at 120° C. for 4 h and calcined at 500° C. for 4 h with a ramp rate of 5° C./min to yield approximately 8 g of product. Neutron activation analysis reveals a total chlorine concentration of 5.8 wt %.

The catalyst is evaluated using a similar procedure as in Example 2. Test conditions and results are shown in Table 3.

TABLE 3

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 1.32% | 37.89% | 3.84% | 56.56% | 0.00% | 0.21% | 1.50% |
| Feed: PhOH | | | | | | | |
| ToS = 1.75 hrs | | | | | | | |
| WHSV 1 hr⁻¹ | | | | | | | |
| T = 500° C. | 0.71% | 37.84% | 5.95% | 51.32% | 0.00% | 0.38% | 4.51% |
| Feed: PhOH | | | | | | | |
| ToS = 2.75 hrs | | | | | | | |
| WHSV 1 hr⁻¹ | | | | | | | |
| T = 500° C. | 0.71% | 52.64% | 6.67% | 40.37% | 0.00% | 0.00% | 0.32% |
| Feed: PhOH | | | | | | | |
| ToS = 4 hrs | | | | | | | |
| WHSV 1 hr⁻¹ | | | | | | | |

### Example 5

A 1M SmCl₃ solution, prepared by dissolving 18.254 g SmCl₃ in 50 ml DI H₂O, is added dropwise along with tetrapropylammonium hydroxide (76.288 g, from a 40 wt % TPAOH solution) over 15 min into a 600 ml beaker containing an initial 100 ml DI H₂O. The solution is stirred at 500 rpm on a magnetic stir plate with a 3 inch stir bar. The resulting precipitate is allowed to age in solution for 1 h with stirring, after which it is centrifuged at 5000 rpm for 10 min. The decanted precipitate is placed into an oven, dried at 120° C. for 4 h and calcined at 500° C. for 4 h with a ramp rate of 5° C./min to yield the solid product.

### Example 6

The catalyst is evaluated using a similar procedure as in Example 2. Test conditions and results are shown in Table 4.

TABLE 4

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 6.08% | 92.99% | 0.02% | 6.99% | 0.00% | 0.00% | 0.00% |
| Feed: PhOH | | | | | | | |
| ToS = 1.75 h | | | | | | | |
| WHSV 1 h⁻¹ | | | | | | | |
| T = 500° C. | 5.36% | 92.16% | 0.90% | 6.93% | 0.00% | 0.00% | 0.00% |
| Feed: PhOH | | | | | | | |
| ToS = 2.75 h | | | | | | | |
| WHSV 1 h⁻¹ | | | | | | | |
| T = 500° C. | 5.16% | 91.91% | 1.49% | 6.60% | 0.00% | 0.00% | 0.00% |
| Feed: PhOH | | | | | | | |
| ToS = 5 h | | | | | | | |
| WHSV 1 h⁻¹ | | | | | | | |
| T = 500° C. | 4.91% | 92.72% | 0.03% | 7.24% | 0.00% | 0.00% | 0.00% |
| Feed: PhOH | | | | | | | |
| ToS = 7 h | | | | | | | |
| WHSV 1 h⁻¹ | | | | | | | |

### Example 7

A 1M GdCl₃ solution, prepared by dissolving 18.633 g GdCl₃ in 50 ml DI H₂O, is added dropwise along with tetrapropylammonium hydroxide (76.261 g, from a 40 wt % TPAOH solution) over 15 min into a 600 ml beaker containing an initial 100 ml DI H₂O. The solution is stirred at 500 rpm on a magnetic stir plate with a 3 inch stir bar. The resulting precipitate is allowed to age in solution for 1 h with stirring, after which it is centrifuged at 5000 rpm for 10 min. The decanted precipitate is placed into an oven, dried at 120° C. for 4 h and calcined at 500° C. for 4 h with a ramp rate of 5° C./min to yield the solid product.

The catalyst is evaluated using a similar procedure as in Example 2. Test conditions and results are shown in Table 5.

TABLE 5

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 16.53% | 92.44% | 0.03% | 6.96% | 0.13% | 0.25% | 0.19% |
| Feed: PhOH | | | | | | | |
| ToS = 1 h | | | | | | | |
| WHSV 1 h⁻¹ | | | | | | | |
| T = 500° C. | 11.05% | 94.78% | 0.50% | 4.21% | 0.11% | 0.20% | 0.21% |
| Feed: PhOH | | | | | | | |
| ToS = 2.75 h | | | | | | | |
| WHSV 1 h⁻¹ | | | | | | | |
| T = 500° C. | 9.44% | 94.64% | 0.82% | 3.85% | 0.13% | 0.17% | 0.38% |
| Feed: PhOH | | | | | | | |
| ToS = 3.5 h | | | | | | | |
| WHSV 1 h⁻¹ | | | | | | | |
| T = 500° C. | 7.09% | 96.00% | 0.22% | 3.36% | 0.04% | 0.11% | 0.26% |
| Feed: PhOH | | | | | | | |
| ToS = 4.75 h | | | | | | | |
| WHSV 1 h⁻¹ | | | | | | | |

### Example 8

The synthesis of chlorinated holmium oxide (Cl—Ho₂O₃) is carried out by a thermal decomposition of HoCl₃.6H₂O. Thus, a sample of the powdered precursor (approximately 10 g) is calcined in air in a static calcination oven under the following temperature protocol: ramp 1.41° C./min to 550° C., dwell 3 hours at 550° C., cool down to room temperature. The chlorine content of the catalyst is assayed by XRF to 13.58 wt. % chlorine. The XRD data shows the presence of holmium oxychloride phases.

The catalyst is used in the dehydration of phenol using a similar procedure as in Example 2. Test conditions and results are shown in Table 6.

TABLE 6

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 1.23% | 92.68% | 0.31% | 7.01% | 0.00% | 0.00% | 0.00% |
| Feed: PhOH | | | | | | | |
| ToS = 2.25 hrs | | | | | | | |
| WHSV 1 hr⁻¹ | | | | | | | |
| T = 500° C. | 1.36% | 91.33% | 0.31% | 8.37% | 0.00% | 0.00% | 0.00% |
| Feed: PhOH | | | | | | | |
| ToS = 3.25 hrs | | | | | | | |
| WHSV 1 hr⁻¹ | | | | | | | |
| T = 500° C. | 1.57% | 88.91% | 0.17% | 10.91% | 0.00% | 0.00% | 0.00% |
| Feed: PhOH | | | | | | | |
| ToS = 4.25 hrs | | | | | | | |
| WHSV 1 hr⁻¹ | | | | | | | |
| T = 500° C. | 1.45% | 87.46% | 0.40% | 12.14% | 0.00% | 0.00% | 0.00% |
| Feed: PhOH | | | | | | | |
| ToS = 5.5 hrs | | | | | | | |
| WHSV 1 hr⁻¹ | | | | | | | |

### Example 9

A 1M DyCl₃ solution, prepared by dissolving 18.849 g DyCl₃ in 50 mL DI H₂O, is added dropwise along with tetrapropylammonium hydroxide (76.261 g, from a 40 wt % TPAOH solution) over 15 min into a 600 mL beaker containing an initial 100 mL DI H₂O. The solution is stirred at 500 rpm on magnetic stir plate with a 3 inch stir bar. The resulting precipitate is allowed to age in solution for 1 h with stirring, after which it is centrifuged at 5000 rpm for 10 min. The decanted precipitate is placed into an oven, dried at 120° C. for 4 h and calcined at 500° C. for 4 h with a ramp rate of 5° C./min to yield 8.6 g of product.

The catalyst is evaluated using a similar procedure as in Example 2. Test conditions and results are shown in Table 7.

TABLE 7

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 8.30% | 96.11% | 0.01% | 3.61% | 0.00% | 0.14% | 0.14% |
| Feed: PhOH | | | | | | | |
| ToS = 1.25 hrs | | | | | | | |
| WHSV 1 hr⁻¹ | | | | | | | |
| T = 500° C. | 11.74% | 97.58% | 0.10% | 2.14% | 0.00% | 0.08% | 0.09% |
| Feed: PhOH | | | | | | | |
| ToS = 2.25 hrs | | | | | | | |
| WHSV 1 hr⁻¹ | | | | | | | |
| T = 500° C. | 6.36% | 96.68% | 0.25% | 2.79% | 0.00% | 0.11% | 0.16% |
| Feed: PhOH | | | | | | | |
| ToS = 3.75 hrs | | | | | | | |
| WHSV 1 hr⁻¹ | | | | | | | |
| T = 500° C. | 11.88% | 95.96% | 0.10% | 3.63% | 0.04% | 0.09% | 0.19% |
| Feed: PhOH | | | | | | | |
| ToS = 4.75 hrs | | | | | | | |
| WHSV 1 hr⁻¹ | | | | | | | |

### Example 10

A 1M YbCl₃ solution, prepared by dissolving 19.387 g YbCl₃ in 50 mL DI H₂O, is added dropwise along with tetrapropylammonium hydroxide (76.265 g, from a 40 wt % TPAOH solution) over 15 min into a 600 mL beaker containing an initial 100 mL DI H₂O. The solution is stirred at 500 rpm on magnetic stir plate with a 3 inch stir bar. The resulting precipitate is allowed to age in solution for 1 h with stirring, after which it is centrifuged at 5000 rpm for 10 min. The decanted precipitate is placed into an oven, dried at 120° C. for 4 h and calcined at 500° C. for 4 h with a ramp rate of 5° C./min to yield 9 g of product.

The catalyst is evaluated using a similar procedure as in Example 2. Test conditions and results are shown in Table 8.

TABLE 8

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 19.76% | 97.39% | 0.15% | 2.18% | 0.02% | 0.19% | 0.07% |
| Feed: PhOH | | | | | | | |
| ToS = 1.5 hrs | | | | | | | |
| WHSV 1 hr⁻¹ | | | | | | | |
| T = 500° C. | 22.20% | 97.53% | 0.06% | 2.20% | 0.02% | 0.14% | 0.06% |
| Feed: PhOH | | | | | | | |
| ToS = 2.5 hrs | | | | | | | |
| WHSV 1 hr⁻¹ | | | | | | | |
| T = 500° C. | 22.29% | 97.55% | 0.10% | 2.10% | 0.04% | 0.14% | 0.06% |
| Feed: PhOH | | | | | | | |
| ToS = 3 hrs | | | | | | | |
| WHSV 1 hr⁻¹ | | | | | | | |

### Example 11

A 1M ErCl₃ solution, prepared by dissolving 15.272 g ErCl₃ in 40 mL DI H₂O, is added dropwise along with tetrapropylammonium hydroxide (61.030 g, from a 40 wt % TPAOH solution) over 15 min into a 600 mL beaker containing an initial 100 mL DI H₂O. The solution is stirred at 500 rpm on magnetic stir plate with a 3 inch stir bar. The resulting precipitate is allowed to age in solution for 1 h with stirring, after which it is centrifuged at 5000 rpm for 10 min. The decanted precipitate is placed into an oven, dried at 120° C. for 4 h and calcined at 500° C. for 4 h with a ramp rate of 5° C./min to yield 7.4 g of product.

The catalyst is evaluated using a similar procedure as in Example 2. Test conditions and results are shown in Table 9.

TABLE 9

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 23.67% | 97.71% | 0.14% | 1.99% | 0.02% | 0.10% | 0.04% |
| Feed: PhOH | | | | | | | |
| ToS = 4.5 hrs | | | | | | | |
| WHSV 1 hr⁻¹ | | | | | | | |
| T = 500° C. | 22.86% | 97.75% | 0.18% | 1.85% | 0.01% | 0.15% | 0.06% |
| Feed: PhOH | | | | | | | |
| ToS = 5.5 hrs | | | | | | | |
| WHSV 1 hr⁻¹ | | | | | | | |

### Example 12

A 1M HoCl₃ solution, prepared by dissolving 11.388 g HoCl₃ in 30 mL DI H₂O, is added dropwise along with tetrapropylammonium hydroxide (45.759 g, from a 40 wt % TPAOH solution) over 15 min into a 600 mL beaker containing an initial 100 mL DI H₂O. The solution is stirred at 500 rpm on magnetic stir plate with a 3 inch stir bar. The resulting precipitate is allowed to age in solution for 1 h with stirring, after which it is centrifuged at 5000 rpm for 10 min. The decanted precipitate is placed into an oven, dried at 120° C. for 4 h and calcined at 500° C. for 4 h with a ramp rate of 5° C./min to yield 5 g of product.

The catalyst is evaluated using a similar procedure as in Example 2. Test conditions and results are shown in Table 10.

TABLE 10

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 12.22% | 97.77% | 0.21% | 1.87% | 0.02% | 0.07% | 0.07% |
| Feed: PhOH | | | | | | | |
| ToS = 1.75 hrs | | | | | | | |
| WHSV 1 hr⁻¹ | | | | | | | |
| T = 500° C. | 12.73% | 97.90% | 0.28% | 1.63% | 0.00% | 0.13% | 0.07% |
| Feed: PhOH | | | | | | | |
| ToS = 3.75 hrs | | | | | | | |
| WHSV 1 hr⁻¹ | | | | | | | |
| T = 500° C. | 12.34% | 97.95% | 0.24% | 1.61% | 0.02% | 0.11% | 0.05% |
| Feed: PhOH | | | | | | | |
| ToS = 5 hrs | | | | | | | |
| WHSV 1 hr⁻¹ | | | | | | | |
| T = 500° C. | 12.02% | 97.88% | 0.12% | 1.82% | 0.00% | 0.11% | 0.07% |
| Feed: PhOH | | | | | | | |
| ToS = 7 hrs | | | | | | | |
| WHSV 1 hr⁻¹ | | | | | | | |

### Example 13

Preparation of the bulk yttrium oxide catalyst precursor, Y₂O₃. A solution of yttrium nitrate is made by dissolving 80.1 g Y(NO₃)₃. 4H₂O in 800 mL deionized H₂O into a four-liter beaker with an overhead stirrer running at 400 rpm. A white precipitate forms as the pH of the solution is adjusted to 9.0 by adding ammonium hydroxide solution with a concentration of 14.6 mol NH₃/liter. The slurry is transferred to a one liter sealed container and heated at 100° C. for 70 hours. The slurry solution is cooled to room temperature and filtered using vacuum filtration in a Buchner funnel. The solid is dispersed in one liter of H₂O, filtered, dispersed in a second liter of H₂O, and filtered again. The solid is then dried at 110° C. for eighteen hours, then the temperature is increased to 600° C. at a rate of 5° C./min held for four hours, and allowed to cool to room temperature.

Preparation of chloride-activated yttrium oxide using ammonium chloride. A solution of ammonium chloride is made by dissolving 0.0604 g of ammonium chloride in 2.0608 mL deionized H₂O. The ammonium chloride solution is then added to 2.0 g of Y₂O₃ dropwise with constant stirring using a spatula. The sample is then dried in air at 120° C. for four hours and then the temperature is increased to 400° C. with a ramp rate of 5° C./min and held for four hours.

The catalyst is evaluated using a similar procedure as in Example 2. Test conditions and results are shown in Table 11.

TABLE 11

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 11.62% | 96.91% | 0.09% | 3.00% | 0.00% | 0.00% | 0.00% |
| Feed: PhOH | | | | | | | |
| ToS = 1.25 hrs | | | | | | | |
| WHSV 1 hr⁻¹ | | | | | | | |
| T = 500° C. | 6.53% | 96.88% | 0.06% | 3.06% | 0.00% | 0.00% | 0.00% |
| Feed: PhOH | | | | | | | |
| ToS = 2 hrs | | | | | | | |
| WHSV 1 hr⁻¹ | | | | | | | |
| T = 500° C. | 4.16% | 96.63% | 0.13% | 3.24% | 0.00% | 0.00% | 0.00% |
| Feed: PhOH | | | | | | | |
| ToS = 3 hrs | | | | | | | |
| WHSV 1 hr⁻¹ | | | | | | | |
| T = 500° C. | 5.48% | 96.34% | 0.24% | 3.42% | 0.00% | 0.00% | 0.00% |
| Feed: PhOH | | | | | | | |
| ToS = 4 hrs | | | | | | | |
| WHSV 1 hr⁻¹ | | | | | | | |

### Example 14

Preparation of zirconia-supported yttrium oxide precursor. A solution of zirconyl chloride is made by dissolving 161.05 g ZrOCl₂ in 2 L deionized H₂O. Solution is added over 1 hour into a 4 L beaker with an overhead stirrer running at 400 rpm starting with 500 ml deionized H₂O. Ammonium hydroxide solution with a concentration of 14.6 mol NH₃/liter is added as needed to maintain the a pH of 10.0 in the solution. A white precipitate is formed and is separated from the liquid by centrifugation for 45 minutes at 3000 rpm and decanting the liquid. The solids are then redispersed in one liter of 60° C. deionized H₂O and the pH is adjusted to 10.0 using ammonium hydroxide. The solids are then separated again by centrifugation and the washing process is repeated four times. The zirconium oxyhydroxide solids are then dried at 120° C. for eighteen hours. A solution of yttrium nitrate is made by dissolving 0.8443 g of yttrium nitrate to enough water to make a solution that is 1.3 mL. The yttrium nitrate solution is then added dropwise with constant stirring using a spatula to 5.0 g of zirconium oxyhydroxide produced in the previous step. The sample is then dried in air at 110° C. for four hours and then the temperature is increased to 600° C. with a ramp rate of 5° C./min and held for four hours.

Preparation of chloride-activated yttrium oxide using aqueous hydrogen chloride. A solution of hydrogen chloride is made by mixing 0.294 mL HCl (10 mol/L) with 0.126 mL deionized H₂O. The hydrogen chloride solution is then added dropwise with constant stirring using a spatula to 3.0 g of zirconia-supported yttrium oxide precursor prepared using the method above. The sample is then dried in air at 120° C. for four hours and then temperature is increased to 400° C. with a ramp rate of 5° C./min and held for four hours.

The catalyst is evaluated using a similar procedure as in Example 2. Test conditions and results are shown in Table 12.

TABLE 12

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 11.35% | 86.62% | 1.26% | 12.04% | 0.00% | 0.08% | 0.00% |
| Feed: PhOH | | | | | | | |
| ToS = 1.5 hrs | | | | | | | |
| WHSV 1 hr⁻¹ | | | | | | | |
| T = 500° C. | 6.63% | 85.43% | 1.18% | 13.29% | 0.00% | 0.00% | 0.10% |
| Feed: PhOH | | | | | | | |
| ToS = 2 hrs | | | | | | | |
| WHSV 1 hr⁻¹ | | | | | | | |
| T = 500° C. | 7.48% | 83.46% | 0.49% | 15.42% | 0.00% | 0.25% | 0.38% |
| Feed: PhOH | | | | | | | |
| ToS = 2.75 hrs | | | | | | | |
| WHSV 1 hr⁻¹ | | | | | | | |
| T = 500° C. | 8.55% | 81.60% | 1.19% | 16.29% | 0.00% | 0.37% | 0.56% |
| Feed: PhOH | | | | | | | |
| ToS = 3.25 hrs | | | | | | | |
| WHSV 1 hr⁻¹ | | | | | | | |

### Example 15

Preparation of fluoride-activated yttrium oxide using ammonium fluoride. A solution of ammonium fluoride is made by dissolving 0.234 g NH₄F in 2.859 mL deionized H₂O. The ammonium fluoride solution is then added to 3.0 g of bulk yttrium oxide precursor prepared using the method from Example 13 dropwise with constant stirring using a spatula. The sample is then dried in air at 120° C. for four hours and then temperature is increased to 400° C. with a ramp rate of 5° C./min and held for four hours.

The catalyst is evaluated using a similar procedure as in Example 2. Test conditions and results are shown in Table 13.

TABLE 13

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 0.37% | 91.55% | 0.01% | 8.44% | 0.00% | 0.00% | 0.00% |
| Feed: PhOH | | | | | | | |
| ToS = 2.25 hrs | | | | | | | |
| WHSV 1 hr⁻¹ | | | | | | | |
| T = 500° C. | 0.23% | 92.55% | 0.02% | 7.43% | 0.00% | 0.00% | 0.00% |
| Feed: PhOH | | | | | | | |
| ToS = 3.5 hrs | | | | | | | |
| WHSV 1 hr⁻¹ | | | | | | | |
| T = 500° C. | 0.32% | 91.81% | 0.18% | 8.01% | 0.00% | 0.00% | 0.00% |
| Feed: PhOH | | | | | | | |
| ToS = 5 hrs | | | | | | | |
| WHSV 1 hr⁻¹ | | | | | | | |
| T = 500° C. | 0.25% | 91.59% | 0.04% | 8.38% | 0.00% | 0.00% | 0.00% |
| Feed: PhOH | | | | | | | |
| ToS = 6.5 hrs | | | | | | | |
| WHSV 1 hr⁻¹ | | | | | | | |

### Example 16

A 1M YCl₃ solution, prepared by dissolving 100.020 g YCl₃ in 330 mL DI H₂O, is added dropwise along with tetrapropylammonium hydroxide (392 mL, from a 40 wt % TPAOH solution) over 15 min into a 2 L beaker containing an initial 500 mL DI H₂O. The solution is stirred at 400 rpm with a 6 mm PTFE screw propeller blade. The resulting precipitate is allowed to age in solution for 3 h with stirring, after which it is centrifuged at 5000 rpm for 10 min. The decanted precipitate is placed into an oven, dried at 120° C. for 4 h and calcined at 500° C. for 4 h with a ramp rate of 5° C./min to yield the solid product.

The catalyst is used for the dehydration of phenol. The powder is pressed and sieved to obtain particles that are between 0.60 mm and 0.85 mm in diameter. The particles are loaded into an electrically heated stainless steel reactor tube and heated to the reaction temperature with nitrogen flowing through the tube. After the reaction temperature is reached, vapor-phase phenol is passed through the reactor tube. The conversion of phenol is carried out at a weight hourly space velocity of 1 (WHSV=gram phenol/gram catalyst-hour) and at 500° C. After 78 h, and about 53% loss in activity, the catalyst is regenerated using the following protocol: the reactor is purged with 50 mL/min of flowing nitrogen for two hours at 500° C., the reactor is then cooled to 300° C. and then a flow of 50 mL/min monochloroethane is passed over the catalyst for 5 minutes and then back to nitrogen flow to purge out the monochloroethane gas. The temperature is then increased to 500° C. and treated with a mixture of 50 mL/min dry air and 100 mL/min nitrogen for four hours. Vapor-phase phenol is then once again passed through the reactor tube. After regeneration, catalyst activity has been fully regained. Test results are shown in Table 14.

| Time on Stream (h) | Phenol Conversion (%) | DPO Selectivity (%) |
|---|---|---|
| 4.5 | 11.46 | 95.56 |
| 23.75 | 10.23 | 96.58 |
| 30.75 | 9.91 | 96.57 |
| 47.5 | 8.00 | 96.55 |
| 55.5 | 5.14 | 96.29 |
| 77 | 5.37 | 95.64 |

Regeneration performed after 78 h according to protocol described above

| | | |
|---|---|---|
| 80 | 11.41 | 88.66 |
| 81.25 | 9.43 | 91.65 |
| 82.5 | 9.11 | 93.11 |
| 84 | 9.04 | 93.43 |

### Example 17

Preparation of the bulk yttrium oxide catalyst precursor, Y₂O₃. A solution of yttrium nitrate is made by dissolving 80.1 g Y(NO3)3.4H2O in 800 mL deionized H₂O into a 4-L beaker with an overhead stirrer running at 400 rpm. A white precipitate forms as the pH of the solution is adjusted to 9.0 by adding ammonium hydroxide solution with a concentration of 14.6 mol NH3/liter. The slurry is transferred to a 1-L sealed container and heated at 100° C. for 70 hours. The slurry solution is cooled to room temperature and filtered using vacuum filtration in a Buchner funnel. The solid is dispersed in one liter of H2O, filtered, dispersed in a second liter of H2O, and filtered again. The solid is then dried at 110° C. for 18 h, then the temperature is increased to 600° C. at a rate of 5° C./min held for four hours, and allowed to cool to room temperature.

Chlorinated yttrium oxide catalyst is prepared from the bulk yttrium oxide by reaction with monochloroethane as follows. The yttrium oxide powder is pressed and sieved to obtain particles that are between 0.60 mm and 0.85 mm in diameter. 5.0 grams of particles are loaded into an electrically heated stainless steel reactor tube and heated to the 300° C. in flowing nitrogen. The flowing gas is then changed to 50 mL/min of monochloroethane for 14 minutes and then back to nitrogen flow to purge out the monochloroethane gas.

For phenol dehydration, the temperature is then increased to 500° C. and the reactor treated with a mixture of 50 mL/min dry air and 100 mL/min nitrogen for four hours. After purging the reactor with nitrogen, vapor-phase phenol is passed through the reactor tube. The conversion of phenol is carried out at a weight hourly space velocity of 0.2 (WHSV=gram phenol/gram catalyst·hour) and at 500° C.

After 84 h, and a loss in activity and selectivity, the catalyst is regenerated using the following protocol: the reactor is purged with 50 mL/min of flowing nitrogen for two hours at 500° C., the reactor is then cooled to 300° C. and then a flow of 50 mL/min monochloroethane is passed over the catalyst for 12 minutes and then back to nitrogen flow to purge out the monochloroethane gas. The temperature is then increased to 500° C. and treated with a mixture of 50 mL/min dry air and 100 mL/min nitrogen for four hours. Vapor-phase phenol is then once again passed through the reactor tube at 500° C. and 0.2 WHSV. After regeneration, catalyst selectivity has been recovered and the catalyst activity is higher than the initial activity. The WHSV is then increased to 0.4 WHSV where the conversion now matches the initial conversion.

| | Reaction | | Phenol | DPO |
|---|---|---|---|---|
| Time on | Temperature | WHSV | Conversion | Selectivity |
| Stream (h) | (° C.) | (g PhOH/g cat-hr) | (%) | (%) |
| 7.75 | 500 | 0.2 | 23.35 | 94.16 |
| 11.25 | 500 | 0.2 | 23.20 | 94.02 |
| 76.75 | 525 | 0.2 | 11.06 | 70.02 |
| 83.25 | 525 | 0.2 | 9.40 | 69.29 |

Regeneration performed after 84 h according to protocol described above.

| | | | | |
|---|---|---|---|---|
| 92 | 500 | 0.2 | 59.73 | 90.70 |
| 94 | 500 | 0.4 | 22.04 | 87.94 |
| 97 | 500 | 0.4 | 23.48 | 90.86 |

1. A method for preparing a diaryl ether compound, the method comprising:
providing a reaction vessel having loaded therein a dehydration catalyst comprising a halogenated rare earth element oxide;
dehydrating an aromatic alcohol compound over the dehydration catalyst to form a diaryl ether compound; and
regenerating the dehydration catalyst by halogenating it with a halogen source.

2. The method of claim 1 wherein the dehydration catalyst is further regenerated through an oxidative treatment step by being heated at elevated temperature in the presence of a gas containing oxygen.

3. The method of claim 1 wherein the halogen source provides chlorine atoms or fluorine atoms.

4. The method of claim 1 wherein the rare earth element oxide is an oxide of a light rare earth element, an oxide of a medium rare earth element, an oxide of a heavy rare earth element, an oxide of yttrium, or mixtures of two or more thereof.

5. The method of claim 1 wherein the rare earth element oxide is an oxide of lanthanum, praseodymium, neodymium, samarium, europium, gadolinium, terbium, dysprosium, holmium, erbium, thulium, ytterbium, lutetium, yttrium, or mixtures of two or more thereof.

6. The method of claim 1 wherein the rare earth element oxide is an oxide of yttrium.

7. The method of claim 1 wherein the dehydration of the aromatic alcohol compound is conducted at a temperature from 200 to 800° C.

8. The method of claim 1 wherein the aromatic alcohol compound is phenol and the diaryl ether produced is diphenyl oxide.

9. The method of claim 1 wherein the diaryl ether compound is recovered through use of condensation, distillation, crystallization, simulated moving bed technique or a combination thereof.

10. The method of claim 1 wherein the diaryl ether compound is recovered through use of one or more of distillation towers or flash vessels.

11. The method of claim 1 wherein unreacted aromatic alcohol is recovered and recycled back to the reactor.

12. (canceled)

13. (canceled)

14. (canceled)

15. (canceled)
